SEQUENCE LISTING (6 sequences)

SEQ ID NO: 1 - DNA, 344 bp, Homo sapiens
cgacccctgtggaccagatgcccagctagtggcctttctccagcccctcagatgacacagaactacaaaccccagcatgcactctggcctgaagtgcctggagagtgctggtgtaccccacctgcattctgggaactgtagtttccctagtcccccatgctcccaccagggcatcaagctcttccctggctggctgaccctgcctcagccctagtctctctgctgactgcggccccgggaagcgtgcgtcactgaatgacagggtgggggtggaggcactcggaaggcagcttcctgctcttttgtgtcccccacttgagtcatgggggtgtgggggttccagg

SEQ ID NO: 2 - DNA, 514 bp, Homo sapiens
gggggactgccccaccctcagcacccaggggaacctcagcccagtagtgaagacctggttatcaggccctatggtagtgccttggctggaggaggggaaagaagtctagacctgctgcaggggtgaggaagtctagacctgctgcaggggtgaggaagtctagacctgctgcaggggtgaggaagtctagacctgctgcgggggtgaggaagtctagacctgctgcgggggtgaggacagctgagcggagttccctgggcggtgctgtcagtagcaggagcagcctcctggaaaagccctggctgctgcttctcccccaagagagaaggcttctcccgccaggccagtccagtgcagcccctcacccacacccactgctaccccagttcccctgcttcggcccgcaccctccctcacaccccagcccacagactcggggctggccttagttactggaacgcctgtgaccacagcactaagagaagcaagctgccccatgggggacttggtcccc

SEQ ID NO: 3 - DNA, 23 bp, Artificial Sequence
  Synthetic Oligonucleotide
cgacccctgtggaccagatgccc

SEQ ID NO: 4 - DNA, 23 bp, Artificial Sequence
  Synthetic Oligonucleotide
cctggaacccccacacccccatg

SEQ ID NO: 5 - DNA, 24 bp, Artificial Sequence
  Synthetic Oligonucleotide
gggggactgccccaccctcagcac

SEQ ID NO: 6 - DNA, 23 bp, Artificial Sequence
  Synthetic Oligonucleotide
ggggaccaagtcccccatggggc

(19) United States
(12) Patent Application Publication
(10) Pub. No.: US 2010/0184072 A1
(43) Pub. Date: Jul. 22, 2010

(54) METHOD OF DETECTION OF PREDISPOSITION TO EMPHYSEMA IN CHRONIC OBSTRUCTIVE PULMONARY DISEASE

(75) Inventors: Mohammad Abdul Qadar Pasha, Delhi (IN); Aarif Ahsan, Delhi (IN)

Correspondence Address: MERCHANT & GOULD PC, P.O. BOX 2903, MINNEAPOLIS, MN, 55402-0903, US

(73) Assignee: COUNCIL OF SCIENTIFIC & INDUSTRIAL RESSEARCH (CSIR), New Delhi (IN)

(21) Appl. No.: 12/694,316

(22) Filed: Jan. 27, 2010

Related U.S. Application Data
(62) Division of application No. 11/004,263, filed on Dec. 2, 2004.

(51) Int. Cl. C12Q 1/68

(52) U.S. Cl. 435/6

(57) ABSTRACT

The present invention relates to a method of detection of predisposition to emphysema in chronic obstructive pulmonary disease (COPD). It particularly relates with the regulation of the key molecular and biochemical components of the pathway leading to the manifestation of emphysema in COPD.

This application is a Divisional of U.S. Ser. No. 11/004,263, filed 2 Dec. 2004, and which application is incorporated herein by reference. To the extent appropriate, a claim of priority is made to the above disclosed application.

### FIELD OF THE INVENTION

The present invention relates to a method of detection of predisposition to emphysema in chronic obstructive pulmonary disease (COPD). It particularly relates with the regulation of the key molecular and biochemical components of the pathway leading to the manifestation of emphysema in COPD.

### BACKGROUND AND PRIOR ART

Chronic Obstructive Pulmonary Disease (COPD) is a detrimental disorder that causes exorbitant human suffering (Snider G 1989). The World Health Organization predicts that by 2020, COPD will rise from its current ranking as the 12th most prevalent disease worldwide to the 5th and from the 6th most common cause of death to the 3rd (Mannino D M 2002). The most important risk factor for COPD is cigarette smoking (Snider G 1989). However, it has been estimated that only 10% of chronic heavy smokers develop symptomatic COPD suggesting that genetic factors are likely to be an important determinant of which cigarette smokers are at risk (Bascpne R 1999). Observed differences in clinical presentations and severity of the disease between racial and ethnic groups together with familial clustering favor a significant hereditary predisposition to the disease (Cohen B H et al 1977). It is more likely that genetic variants reflecting in difference in biochemical parameters are associated with the disease. COPD is characterized mainly by the three pathophysiological ailments, namely endothelial dysfunction, inflammation and chronic bronchitis that lead to emphysema (Morrison D et al 1999, Barnes P J 2000, Dinh Xuan A T 1991, Saetta M 1999). Along with that oxidant/antioxidant imbalance in favor of oxidants impart oxidative stress in the disease (Sanguinetti C M 1992). The oxidative burden produced by inhaling cigarette smoke can be further enhanced in the lungs by the release of oxygen radicals from the influx and activation of inflammatory leukocytes (Heidal J R et al 1981). Intracellular enzymes like NADPH oxidase and endothelial nitric oxide synthase (eNOS) by producing superoxide radical and nitric oxide respectively play a major role to increase the overall oxidative stress of the system. The generation of oxidants in epithelial lining fluid in smokers is further enhanced by the presence of increased amounts of free iron in the airspaces (Mateos F et al 1998). Free iron in the ferrous form can take part in the Fenton and Haber-Weiss reactions, which generate the hydroxyl radical, a free radical that is extremely damaging to all tissues, particularly to cell membranes, producing lipid peroxidation (Mateos F et al 1998). Markers of oxidative stress have been demonstrated in the epithelial lining fluid, in the breath and in the urine in cigarette smokers and patients with COPD, and there has been interest in evidence of systemic oxidative stress, measured in the blood (Cross C E et al 1993). Alveolar leukocytes from smokers and patients with chronic bronchitis have increased ability to release oxygen radicals, compared with those from healthy controls (Heidal J R et al 1981). Components of lung matrix (such as elastin and collagen) can be directly damaged by oxidants to cause emphysema, a major hallmark of COPD (Cantin A et al 1985). Apart from oxidative stress, inactivation of antioxidant capacity of the system aggravates the formation of emphysematous spaces of the lung tissue. There is now overwhelming evidence of the increment of oxidative stress by a fall in the anti-oxidant capacity of blood in COPD patients (Rahman I et al 1996). The decrease in antioxidant capacity in the COPD patients could be due to depletion to a number of factors in the plasma, including protein sulfadryls, which are depleted after exposure of plasma to cigarette smoke in vitro. Other investigators have shown that other major plasma antioxidant such as ascorbic acid, vitamin E, β-carotene and selenium are depleted in the serum of COPD patients (MacNee W et al 1999, Rahman I et al 1996). Endogenous antioxidant enzyme catalase plays an important role in scavenging hydrogen peroxide, one of the highly reactive oxidative radicals.

The best approach for the dissection of complex disorders like COPD would be a multidimensional approach involving major hallmarks of the disease. The approach will include components of the main pathways in the disease and connecting them to present a better understanding of the disease. In this context, COPD is characterized by three pathophysiological ailments, endothelial dysfunction, inflammation and chronic bronchitis. These three processes culminate into emphysema in the disease.

### Current Status of the Treatment of COPD:

1. Smoking cessation: Smoking cessation reduces rates of decline in lung function in COPD patients. A prime example of this possibility was the finding that smoking cessation for six months reduced the numbers of alveolar macrophages and neutrophils recoverable by lung lavage (Turato G et al 1995).
2. Sympathomimetics/Anticholinergics: While, the primary effect of β₂ agonists is to relax airway smooth muscle, some antioxidant benefit may be provided as well. For example, superoxide radical formation by macrophages was decreased in chronic bronchitis patients treated with formoterol. Likewise, incubating blood neutrophils from healthy volunteers with increasing concentration of theophylline inhibited their oxygen radical production in a dose dependent manner (Llewellyn Jones C G et al 1994, Nielson C P et al 1986).
3. NO therapy: NO has been used as an inhalation therapy for the treatment of COPD. NO can decrease the partial pressure of arterial oxygen in patients with COPD. It exerts its effect mainly via improvement of ventilation/perfusion ratio and lowering of alveolar to arterial oxygen tension difference by increasing arterial oxygen saturation (Mofuard J et al 1994).
4. Antioxidant therapy: N-acetylcysteine (NAC) is the most widely investigated drug with antioxidant properties that has been used in both experimental and clinical settings, which are relevant to COPD. NAC is a thiol-containing compound that may act as an antioxidant by providing cysteine intracellularly for the enhanced production of cysteine intracellularly for the enhanced production of GSH (Cross C E et al 1984, Moldeus P et al 1986).
5. Corticosteroids: Steroids have an antioxidant effect by decreasing the numbers as well as the oxidative and chemotactic responses of neutrophils (Renkema T E J et al 1993).
6. Genetic predisposition: Variations in a number of genes have been found to be associated with the prevalence of COPD. For example, people with ZZ phenotype of α1-Antitrypsin have a clearly accelerated rate of decline in lung function, and the proportion of GSTP1 homozygotes of Glutathione-S-transferase was significantly higher in COPD patients (Brantly M L et al 1988, Ishii T et al 1999).

### Limitations of the Available Therapies for COPD:

1. Sometimes the patients do not respond to smoking cessation, as amelioration of the disease becomes impossible at these stages.
2. The effects of Sympathomimetics/Anticholinergics like theophylline on oxygen radical generation by neutrophils remain controversial. In few studies, theophylline treatment enhanced superoxide radical production by neutrophils (Kaneko M et al 1990).
3. COPD patients do not found to have homogenous response to NO inhalation. Moreover, concentration of required NO varies with the severity of the disease. In few COPD patients, hypoxemia is caused essentially by ventilation/perfusion imbalance rather than by shunt, inhaled NO worsens gas exchange because of impaired hypoxia regulation of the matching between ventilation and perfusion (Mofuard J et al 1994).
4. Antioxidants like NAC were also found to produce normal amounts of hydrogen peroxide from neutrophils (Linden M et al 1988).
5. Corticosteroids like Dexamethasone are non-selective immune suppressors. However, in numerous cases it was not found to correlate with its immune suppression activity. For example, in one study dexamethasone did not alter unstimulated superoxide radical production by neutrophils either in vitro or in vivo (Lomas D A et al 1991). Likewise, inhaled corticosteroids did not change neutrophil numbers or IL-8 levels in the peripheral blood of COPD patients (DeBcuker W A et al 1996).
6. Despite the association of polymorphims in genes with COPD, the data remains conflicting as highly significant polymorphisms like ZZ homozygote of α1-Antitrypsin also correlated with normal lung function (Silverman E K et al 1989).

Novelty of the present invention is in providing a method of detection of predisposition to emphysema in COPD.

Still another novelty is to provide a novel marker region containing the COPD associated -786T/C polymorphism in the eNOS gene.

Still another novelty is to provide a novel marker region containing the COPD associated 4B/4A polymorphism in the eNOS gene.

Still another novelty is to provide novel primers and probes for the amplification of the novel marker regions, which contain the polymorphisms.

Still another novelty is to demonstrate a significant association of -786C allele of eNOS gene with COPD and -786T allele with the unaffected phenotype.

Still another novelty is to demonstrate a significant association of 4A allele of eNOS gene with COPD and 4B allele with the unaffected phenotype.

Still another novelty is to provide increased endogenous nitrite level as a novel biochemical marker in COPD patients.

Still another novelty is to provide decreased Catalase activity as a novel biochemical marker in COPD patients.

Another novelty is to provide increased lipid peroxidation level as a novel biochemical marker in COPD patients.

### OBJECTS OF THE INVENTION

Main object of the invention is to provide a method of detection of predisposition to emphysema in COPD, which obviates the limitations listed above.

Still another object is to provide novel polymorphisms in the eNOS gene associated with the susceptibility to COPD.

Still another object is to provide novel primers and probes for the amplification of the marker regions containing the polymorphisms.

Still another object is to provide allelic variants of the eNOS gene associated with the susceptibility to COPD.

Still another object is to provide novel biochemical markers associated with COPD. Yet in another object of the present invention is to develop a kit for detecting COPD associated allelic version.

### SUMMARY OF THE INVENTION

The present invention relates to a method of detection of predisposition to emphysema in chronic obstructive pulmonary disease (COPD). It particularly relates with the regulation of the key molecular and biochemical components of the pathway leading to the manifestation of emphysema in COPD. COPD is characterized mainly by three pathophysiological ailments, namely endothelial dysfunction, inflammation and chronic bronchitis that leads to emphysema. Along with that oxidant/antioxidant imbalance in the favor of oxidants impart oxidative stress in the disease. The present invention relates to a method of detection of predisposition to emphysema in COPD and it comprises of the regulation of key molecular and biochemical components of a pathway leading to the manifestation of emphysema in COPD.

### BRIEF DESCRIPTION OF THE DRAWINGS

The manner in which the above-mentioned features, advantages and objects of the invention, as well as others, which will become clear, are attained and can be understood in detail, by the particular description of the invention are illustrated in the appended drawings. These drawings form a part of the specification. It is to be noted, however, that the appended drawings illustrate preferred embodiments of the invention and thereof not to be considered limiting in their scope.

FIG. 1 shows genotype distribution of -786T/C conversion in controls and patients

FIG. 2 shows genotype distribution of 4B/4A conversion in controls and patients

FIG. 3 shows plasma NO levels between the controls and patients

FIG. 4 shows plasma Catalase activity between the controls and patients

FIG. 5 shows Plasma LPO levels between the controls and patients

Table 1 Summary of the molecular and biochemical differences observed between controls and patients

### DETAILED DESCRIPTION OF THE PRESENT INVENTION

Accordingly, the present invention relates to the components of a novel pathway for the detection of predisposition to emphysema in COPD. It particularly relates with the elucidation of molecular and biochemical basis of emphysema in COPD.

In one embodiment of the present invention a method of detection of predisposition to emphysema in chronic obstructive pulmonary disease (COPD), the said method comprises the steps:
a. selecting study subjects by monitoring COPD associated phenotypes;
b. extracting genomic DNA from leukocytes of the subject by known methods;
c. computationally locating the marker regions -786T/C and 4B/4A on the eNOS gene of the genomic DNA containing the polymorphisms in the eNOS gene as given in SEQ ID Nos. 1 and 2,
d. amplifying the polymorphism containing marker regions of the eNOS gene using novel primers as given in SEQ ID Nos. 3, 4, 5 and 6;
e. genotyping the polymorphic product products of step (iv) by restriction digestion and polyacrylamide gel electrophoresis and computing the frequencies of -786T, -786C, 4B and 4A alleles; and
f. statistically analyzing the differences in the distribution of the allelic variants -786 T, -786 C, 4B and 4A, wherein -786 T and 4B alleles are associated with low risk and -786 C and 4A alleles at high risk of the disease.

In another embodiment of the present invention a method of detection of predisposition to emphysema in chronic obstructive pulmonary disease wherein the allelic variants of eNOS gene associated with COPD are -786 T, -786 C, 4B and 4A. Still in another embodiment of the present invention the oligonucleotide primers of SEQ. ID NO. 3, 4, 5 and 6 are used for detection.

Yet in another embodiment of the present invention the detection can be performed on a plurality of individuals who are tested either for the presence or for the predisposition to COPD and the susceptibility to the disease can then be established based on the base or set of bases present at the polymorphic sites in the individuals tested.

Further, the invention provides a diagnostic kit comprising primers or probes of SEQ ID Nos. 3, 4, 5 and 6 along with the required buffers, instruction manual and accessories suitable for identification of eNOS allelic variants to establish a subject's susceptibility to COPD.

Further in another embodiment of the present invention wherein the diagnostic kit further comprises restriction enzymes, reverse transcriptase or polymerase, the substrate nucleoside triphosphates, means used to label and the appropriate buffers for reverse transcription, PCR, or hybridization reactions.

Yet in another embodiment of the present invention wherein in the diagnostic kit the label used is biotin.

Yet in another embodiment of the present invention wherein in the diagnostic kit the means for labeling is selected from streptavidin enzyme conjugate and enzyme substrate and chromogen.

Yet in another embodiment of the present invention wherein in the diagnostic kit the nucleic acid vectors used contain the allelic variants of the eNOS gene.

Further in another embodiment of the present invention wherein a method of detection of predisposition to emphysema in chronic obstructive pulmonary disease (COPD) is developed using biochemical markers, wherein the said method comprising the detection of nitrite levels, catalase activity and lipid peroxidation in plasma in the study subject, wherein the elevated level of nitrite and lipid peroxidation and reduced level of catalase activity in plasma indicates the presence of predisposition to COPD.

In one more embodiment of the present invention the nitrite level in the plasma of study subjects is estimated by colorimetric assay.

Yet in another embodiment of the present invention the catalase activity is estimated by H₂O₂ consumption/min/mg of protein in the plasma of the study subjects.

Yet in another embodiment of the present invention the lipid peroxidation level is estimated by measuring Malonaldehyde (MDA) in the plasma.

Yet in another embodiment of the present invention the nitrite level in plasma is elevated significantly wherein the p value is 0.02.

Yet in another embodiment of the present invention the catalase activity in plasma is decreased significantly wherein the p value 0.05.

Yet in another embodiment of the present invention the study subject is mammal and preferably human.

In one more embodiment of the present invention an eukaryotic vectors comprising a DNA sequence coding for a protein or a peptide according to the invention are new materials and are also included in the invention. Host cells, for example, cloned human cell lines, can be transformed using the new vectors and are also included in the invention.

I. Identification of the Marker Regions on the eNOS Gene

Taking into consideration the important functions of NO in vasodilation and improved oxygenation that are generally defective in COPD, the marker regions, -786T/C and 4B/4A in the eNOS gene were selected for the study.

### II. Selection of the Study Subjects

Inclusion criteria for the patients was based on a reduction of both FEV₁ and the FEV₁/FVC ratio. Emphysema related symptoms such as breathlessness, lingering cough (smoker's cough), and morning headaches were also monitored to confirm the disease. All patients with COPD were clinically stable, and none had a history of respiratory infection for at least 4-weeks period preceding the study and no asthma. Patients had a smoking history of at least 10 cigarettes daily for more than ten packed years.

III. Extraction of Genomic DNA from Leukocytes and Separation of Plasma

Genomic DNA was extracted from blood by salting out method. Lysis of red blood cells in presence of high salt was followed by treatment with Nucleus lysis buffer. Proteins were precipitated and extraction of DNA was obtained in ethanol. Plasma samples from study subjects were separated by using an anticoagulant. After centrifugation clear plasma samples were stored at −20° C.

IV. Association Analysis with the Disease:

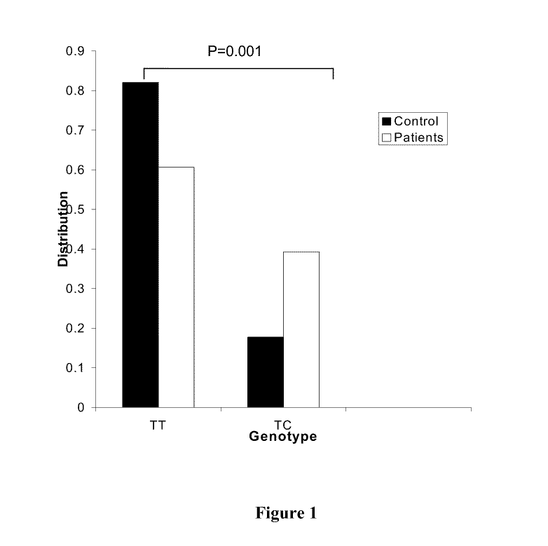

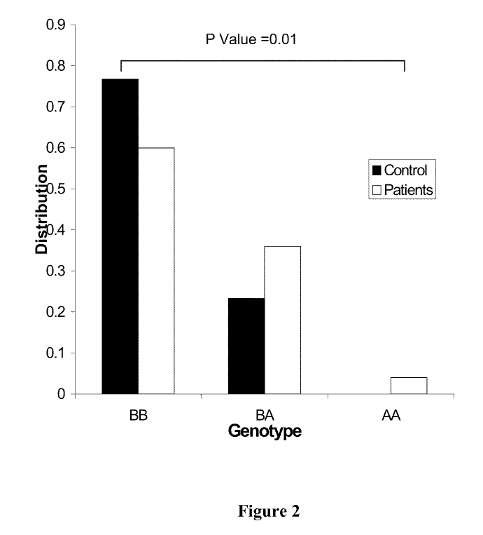

Analysis of both the polymorphisms in 32 COPD patients and 35 controls revealed four alleles, -786T, -786C, 4B and 4A. The distribution of alleles is summarized in FIGS. 1 and 2. The frequency of -786C and 4A alleles were found to be greater in COPD patients than the controls. The biostatistical analysis showed a significant association of -786 C and 4A alleles with the disease as mentioned in FIGS. 1 and 2. One of the pathophysiology of COPD is endothelial dysfunction that mediates pulmonary hypertension in the disease. In the present investigation, the significant over-representation of -786C and 4A alleles of eNOS gene have been observed in the patients as compared to the controls. In conjunction with few other parameters the faulty eNOS containing both the mutant alleles -786C and 4A conferring endothelial dysfunction may result in reduced nitric oxide generation. This can act as a stimulus for the activation of inducible nitric oxide synthase (iNOS).

### V. Diagnostic Kits

The invention further provides diagnostic kit comprising at least one or more allele specific oligonucleotides as described in SEQ ID 1, 2, 3 and 4. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least the polymorphisms studied in the present investigation. Optional additional components of the kit include, for example, restriction enzymes, reverse transcriptase or polymerase, the substrate nucleoside triphosphates, means used to label (for example, an avidin enzyme conjugate and enzyme substrate and chromogen if the label is biotin), and the appropriate buffers for reverse transcription, PCR, or hybridization reactions. Usually, the kit also contains instructions for carrying out the methods.

### VI. Nucleic Acid Vectors

Variant genes can be expressed in an expression vector in which a variant gene is operably linked to a native or other promoter. Usually, the promoter is eukaryotic promoter for expression in a mammalian cell. The transcription regulation sequences typically include a heterologous promoter and optionally an enhancer, which is recognized by the host. The selection of an appropriate promoter, for example trp, lac, phage promoters, glycolytic enzyme promoters and tRNA promoters, depends on the host selected. Commercially available expression vectors can also be used. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide.

The invention further provides transgenic non-human animals capable of expressing an exogenous variant gene and/or having achieved by operably linking the gene to a promoter and optionally an enhancer, and microinjecting the construct into a zygote. Inactivation of endogenous variant genes can be achieved by forming a transgene in which a cloned variant gene is inactivated by insertion of a positive selection marker. The transgene is then introduced in to an embryonic stem cell, where it undergoes homologous recombination with an endogenous variant gene. Mice and other rodents are preferred animals. Such animals provide useful drug screening systems.

### VII. Plasma Nitrite Estimation

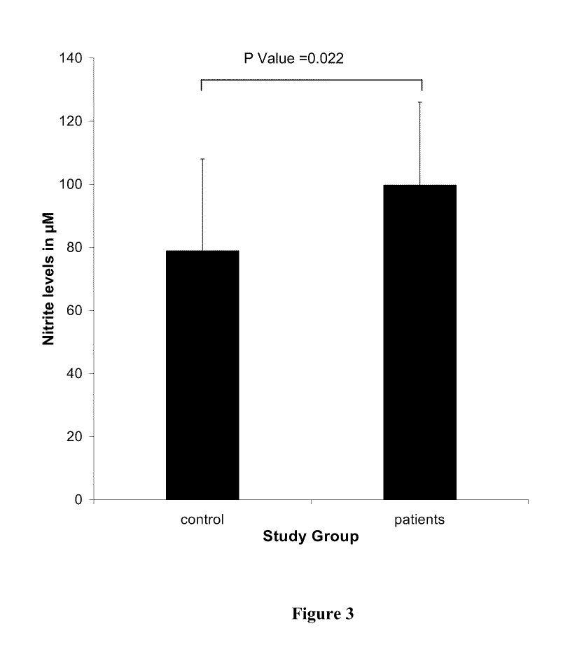

Plasma nitrite levels were estimated in the study subjects. The patients were found to have significantly elevated nitrite levels. Inflammation often accompanies the pathophysiology of COPD, which will activate iNOS in conjunction with already available endothelial dysfunction by faulty eNOS containing both the mutant alleles. This will result in outrageous NO production by the inducible enzyme. NO, being a short-lived molecule immediately gets converted in nitrate and nitrite. The present study involved the method for estimation of total nitrite of the system since the enzyme nitrate reductase converts total nitrate to nitrite. It was found that level of nitrite was significantly elevated in case of patients than the controls. The levels are presented in FIG. 3. On the other hand, inflammation simultaneously may result in recruitment of macrophages. The immune effector cells by respiratory burst will result in the production of superoxide radical (O₂—) by the involvement of an oxidant enzyme NADPH oxidase. Out of the several routes that O₂— can take, it may combine with elevated nitrite in the patients to form increased peroxinitrite radical. Otherwise, it normally gets converted into hydrogen peroxide (H₂O₂) by the action of superoxide dismutase (SOD). Now, H₂O₂ can further be catalyzed by two pathways. It can either be converted into H₂O and O₂ by the action of antioxidant enzyme Catalase, or through Fenton reaction it may be converted into hydroxy radical (OH—).

### VIII. Catalase Activity Assay

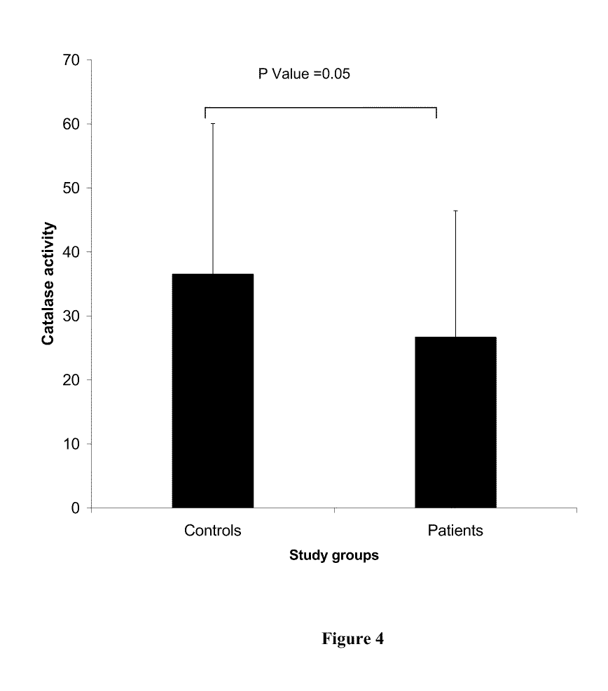

Plasma Catalase activity was measured in study subjects. The activity was calculated in terms of nmoles of H₂O₂ consumed/min/mg of protein. Changes in absorbance were recorded at 240 nm for three minutes so that to calculate per minute decrease in optical density. The results are presented in FIG. 4. We have observed significant decreased activity of Catalase that will result in increased production of hydroxy radical (OH—) because H₂O₂ will take the second route. Hence, increased peroxinitrite via increased nitrite level and O₂— and increased OH— via decreased Catalase activity may result in increased lipid peroxidation of the plasma membrane of the cells.

### IX. Lipid Peroxidation Measurement

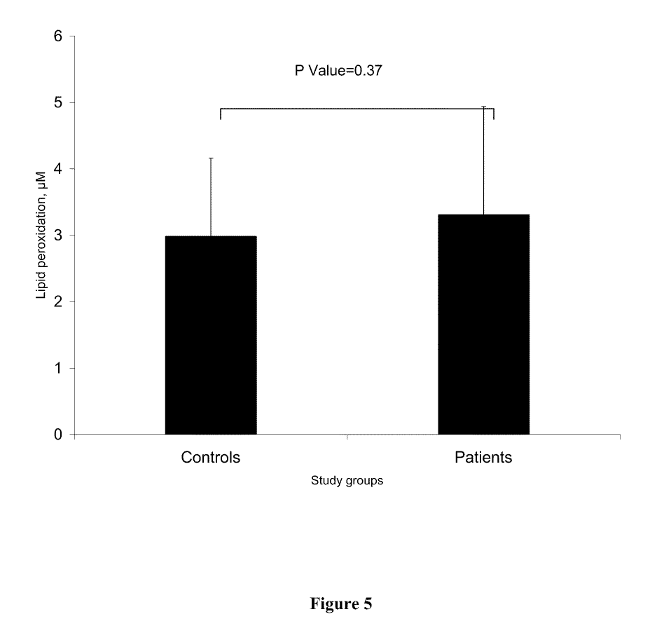

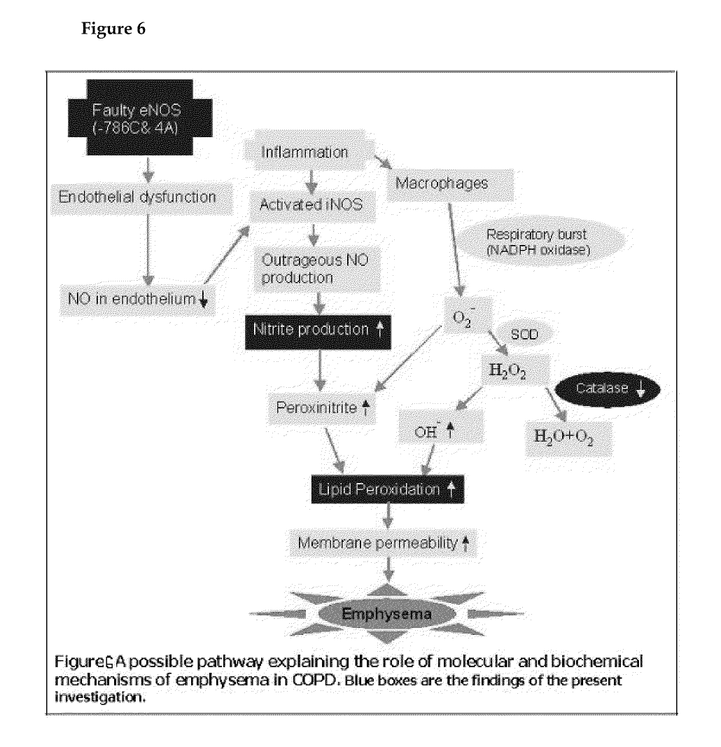

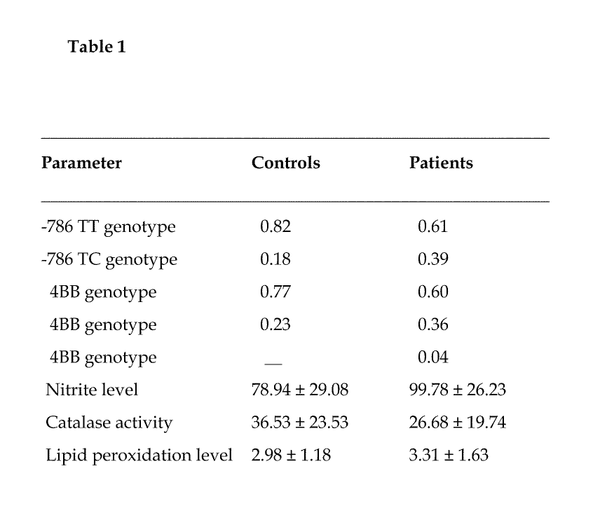

The Lipid peroxidation (LPO) levels were measured in study subjects. The amount of Malondialdehyde (MDA) formed in each of the sample was assayed by measuring the optical density of the supernatant at 535 nm after the precipitation of proteins by Trichloroacetic acid. The results are presented in FIG. 5. We have observed elevated LPO level in the patients as compared to the controls justifying the over-activity of these oxidative species.

The following examples are given by way of illustration of the present invention and should not be construed to limit the scope of the present invention.

### Example 1

### I. Identification of the Marker Regions on the eNOS Gene

Taking in consideration the important functions of NO in vasodilation and improved oxygenation that are generally defective in COPD, the following novel marker regions, containing -786T/C and 4B/4A polymorphisms were selected from the sequence of the eNOS gene (NT_{—}007914) for the study.

−786 T/C polymorphism

(SEQ ID NO: 1)

5′ CGA CCC CTG TGG ACC AGA TGG CCA GCT AGT GGC CTT
TCT CCA GCC CCT CAG ATG ACA CAG AAC TAC AAA CCC
CAG CAT GCA CTC TGG CCT GAA GTG CCT GGA GAG TGC
TGG TGT ACC CCA CCT GCA TTC TGG GAA CTG TAG TTT
CCC TAG TCC CCC ATG CTC CCA CCA GGG CAT CAA GCT
CTT CCC TGG CTG GCT GAC CCT GCC TCA GCC CTA GTC
TCT CTG CTG ACT GCG GCC CCG GGA AGC GTG CGT CAC
TGA ATG ACA GGG TGG GGG TGG AGG CAC T/C*GG AAG GCA
GCT TCC TGC TCT TTT GTG TCC CCC ACT TGA GTC ATG
GGG GTG TGG GGG TTC CAG G 3′
In the above sequence the SNP* is shown in bold.
4B/4A polymorphism

(SEQ ID NO: 2)

5′ GGG GGA CTG CCC CAC CCT CAG CAC CCA GGG GAA CCT
CAG CCC AGT AGT GAA GAC CTG GTT ATC AGG CCC TAT
GGT AGT GCC TTG GCT GGA GGA GGG GAA AGA AGT CTA
GAC CTG CTG CAG GGG TGA GGA AGT CTA GAC CTG CTG
CAG GGG TGA GGA AGT CTA GAC CTG CTG CAG GGG TGA
GGA AGT CTA GAC CTG CTG CGG GGG TGA GGA AGT CTA
GAC CTG CTG CGG GGG TGA GGA CAG CTG AGC GGA GTT
CCC TGG GCG GTG CTG TCA GTA GCA GGA GCA GCC TCC
TGG AAA AGC CCT GGC TGC TGC TTC TCC CCC AAG AGA
GAA GGC TTC TCC CGC CAG GCC AGT CCA GTG CAG CCC
CTC ACC CAC ACC CAC TGC TAC CCC AGT TCC CCT GCT
TCG GCC CGC ACC CTC CCT CAC ACC CCA GCC CAC AGA
CTC GGG GCT GGC CTT AGT TAC TGG AAC GCC TGT GAC
CAC AGC ACT AAG AGA AGC AAG CTG CCC CAT GGG GGA
CTT GGT CCC C 3′

In the above sequence of the variable number of tandem repeat is shown in bold.

### Example 2

### II. Selection of the Study Subjects

Inclusion criteria for the patients based on a reduction of both FEV₁ and the FEV₁/FVC ratio. Emphysema related symptoms such as breathlessness, lingering cough (smoker's cough), and morning headaches were also monitored to confirm the disease. All patients with COPD were clinically stable, and none had a history of respiratory infection for at least 4-weeks period preceding the study and no asthma. Patients had a smoking history of at least 10 cigarettes daily for more than ten packed years.

### Example 3

### III. Extraction of Genomic DNA from Leukocytes and Separation of Plasma

Genomic DNA was extracted from blood using salting out method. Lysis of red blood cells in presence of high salt was followed by treatment with Nucleus lysis buffer. Proteins were precipitated and DNA was extracted from peripheral blood leukocytes using a modification of the salting out procedure. The concentration of the DNA was determined by measuring the optical density of the sample, at a wavelength of 260 nm. Plasma samples from study subjects were separated by using an anticoagulant. After centrifugation clear plasma samples were stored at −20° C.

### Example 4

### IV. Genotyping of the Study Subjects

This example describes the genotyping of allelic variants of eNOS gene. The DNA was amplified by polymerase chain reaction by using the following oligonucleotide primers:

−786 T/C polymorphism
1. 5′ CGA CCC CTG TGG ACC AGA TGC CC 3′ (listed as
SEQ ID NO: 3)
and
2. 5′ CCT GGA ACC CCC ACA CCC CCA TG 3′ (listed as
SEQ ID NO: 4)

Polymerase Chain Reaction was Carried Out Using the Following Conditions:
Step 1 94° C. for 4 min
Step 2 94° C. for 30 sec
Step 3 67.0° C. for 30 sec
Step 4 72° C. for 45 sec
Step 5 34 times repetition of step 2 through 4
Step 6 72° C. for 10 min

4B/4A polymorphism
3. 5′ GGG GGA CTG CCC CAC CCT CAG CAC 3′ (listed
as SEQ ID NO: 5)
and
4. 5′ GGG GAC CAA GTC CCC CAT GGG GC 3′ (listed as
SEQ ID NO: 6)

Polymerase chain reaction was carried out using the following conditions:
Step 1 94° C. for 4 min
Step 2 94° C. for 30 sec
Step 3 68.0° C. for 30 sec
Step 4 72° C. for 45 sec
Step 5 34 times repetition of step 2 through 4
Step 6 72° C. for 10 min

PCR was performed in a Perkin Elmer GeneAmp PCR System 9600. This reaction produced a DNA fragment of 343 by for -786T/C polymorphism and 514 by and 487 by for 4B and 4A alleles of 4B/4A polymorphism. For -786T/C polymorphism Ngo M IV enzyme was used for restriction fragment length polymorphism and for 4B/4A polymorphism both the alleles were resolved by 12% Polyacrylamide gel electrophoresis.

### Example 5

### V. -786C and 4A Alleles are Associated with the Disease

Analysis of both the polymorphisms in 32 COPD patients and 35 controls revealed four alleles, -786T, -786C, 4B and 4A. The distribution of alleles is summarized in FIGS. 1 and 2. The frequency of -786C and 4A alleles were found to be greater in COPD patients than the controls. The biostatistical analysis showed a significant association of -786C and 4A alleles with the disease as mentioned in FIGS. 1 and 2. One of the pathophysiology of COPD is endothelial dysfunction that mediates pulmonary hypertension in the disease. In the present investigation, the significant over-representation of -786C and 4A alleles of eNOS gene have been observed in the patients as compared to the controls. In conjunction with few other parameters the faulty eNOS containing both the mutant alleles -786C and 4A conferring endothelial dysfunction will result in reduced nitric oxide generation. This can act as a stimulus for the activation of inducible nitric oxide synthase (iNOS).

### Example 6

### VI. Diagnostic Kits

The invention further provides diagnostic kit comprising at least one or more allele specific oligonucleotides as described in SEQ ID 1, 2, 3 and 4. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least the polymorphisms studied in the present investigation. Optional additional components of the kit include, for example, restriction enzymes, reverse transcriptase or polymerase, the substrate nucleoside triphosphates, means used to label (for example, an avidin enzyme conjugate and enzyme substrate and chromogen if the label is biotin), and the appropriate buffers for reverse transcription, PCR, or hybridization reactions. Usually, the kit also contains instructions for carrying out the methods.

### Example 7

### VII. Nucleic acid vectors

Variant genes can be expressed in an expression vector in which a variant gene is operably linked to a native or other promoter. Usually, the promoter is eukaryotic promoter for expression in a mammalian cell. The transcription regulation sequences typically include a heterologous promoter and optionally an enhancer, which is recognized by the host. The selection of an appropriate promoter, for example trp, lac, phage promoters, glycolytic enzyme promoters and tRNA promoters, depends on the host selected. Commercially available expression vectors can also be used. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide.

The invention further provides transgenic non-human animals capable of expressing an exogenous variant gene and/or having achieved by operably linking the gene to a promoter and optionally an enhancer, and microinjecting the construct into a zygote. Inactivation of endogenous variant genes can be achieved by forming a transgene in which a cloned variant gene is inactivated by insertion of a positive selection marker. The transgene is then introduced in to an embryonic stem cell, where it undergoes homologous recombination with an endogenous variant gene. Mice and other rodents are preferred animals. Such animals provide useful drug screening systems.

### Example 8

### VIII. Plasma Nitrite Levels were Elevated in the Patients

Plasma nitrite levels were estimated in the study subjects. The patients were found to have significantly elevated nitrite levels. Inflammation often accompanies the pathophysiology of COPD, which will activate iNOS in conjunction with already available endothelial dysfunction by faulty eNOS containing both the mutant alleles. This will result in outrageous NO production by the inducible enzyme. NO, being a short-lived molecule immediately gets converted in nitrate and nitrite. The present study involved the method for estimation of total nitrite of the system since the enzyme nitrate reductase converts total nitrate to nitrite. It was found that level of nitrite was significantly elevated in case of patients than the controls. The levels are presented in FIG. 3. On the other hand, inflammation simultaneously may result in recruitment of macrophages. The immune effector cells by respiratory burst will result in the production of superoxide radical (O₂—) by the involvement of an oxidant enzyme NADPH oxidase. Out of the several routes that O₂— can take, it may combine with elevated nitrite in the patients to form increased peroxinitrite radical. Otherwise, it normally gets converted into hydrogen peroxide (H₂O₂) by the action of superoxide dismutase (SOD). Now, H₂O₂ can further be catalyzed by two pathways. It can either be converted into H₂O and O₂ by the action of antioxidant enzyme Catalase, or through Fenton reaction it may be converted into hydroxy radical (OH—).

### Example 9

### IX. Plasma Catalase Activity was Decreased in the Patients

Plasma Catalase activity was measured in study subjects. The activity was calculated in terms of nmoles of H₂O₂ consumed/min/mg of protein. Changes in absorbance were recorded at 240 nm for three minutes so that to calculate per minute decrease in optical density. The results are presented in FIG. 4. We have observed significant decreased activity of Catalase that will result in increased production of hydroxy radical (OH—) because H₂O₂ will take the second route. Hence, increased peroxinitrite via increased nitrite level and O₂— and increased OH— via decreased Catalase activity may result in increased lipid peroxidation of the plasma membrane of the cells.

### Example 10

### X. Plasma Lipid Peroxidation Level was Elevated in the Patients

The Lipid peroxidation LPO levels were measured in study subjects. The amount of MDA formed in each of the sample was assayed by measuring the optical density of the supernatant at 535 nm after the precipitation of proteins by Trichloroacetic acid. The results are presented in FIG. 5. We have observed elevated lipid peroxidation (LPO) level in the patients as compared to the controls justifying the over-activity of these oxidative species.

### Example 11

### XI. A Novel Pathway for the Detection of Predisposition of Emphysema in COPD

The findings summarized in Table 1 were presented as a novel pathway (FIG. 6) based on the available literature about the disease. The pathway includes the combination of molecular and biochemical mechanisms for a better understanding about the disease.

### ADVANTAGES OF THE PRESENT INVENTION

1. The components of a novel pathway for the detection of predisposition to emphysema in COPD.
2. Novel maker regions containing the -786T/C and 4B/4A polymorphisms.
3. Novel primer sequences responsible for amplification of PCR product containing the polymorphisms.
4. A significant association of -786 C allele of eNOS gene with COPD (FIG. 1).
5. A significant association of 4A allele of eNOS gene with COPD (FIG. 2).
6. A novel biochemical marker of increased endogenous nitrite level in COPD patients (FIG. 3).
7. A novel biochemical marker of decreased Catalase activity in COPD patients (FIG. 4).
8. A novel biochemical marker of increased lipid peroxidation level in COPD patients (FIG. 5).
9. It may help in determining the predisposition of the individuals to the disease.

### REFERENCES

1. Snider, G. Chronic obstructive pulmonary disease: risk factors, pathophysiology and pathogenesis. Annu. Rev. Med. 1989; 40:411-429.
2. Mannino D M. COPD: epidemiology, prevalence, morbidity and mortality, and disease heterogeneity. Chest 2002; 121(5):121S-126S.
3. Bascpne R. Differential susceptibility to tobacco smoke: possible mechanisms. Pharmacogenetics 1999; 1:102-106.
4. Cohen B H, Diamond E L, Graves C G et al. A common familial component in lung cancer and chronic obstructive pulmonary disease. Lancet 1977; 2(8037):523-6.
5. Morrison D, Rahman I, Lannan S and MacNee W. Epithelial permeability inflammation and oxidant stress in the air spaces of smokers. Am. J. Respir. Crit. Care Med. 1999; 159:473-479.
6. Barnes P J. Chronic obstructive pulmonary disease. N. Engl. J. Med. 2000; 343:269-280.
7. Dinh Xuan A T, Higenbottam T W, Clelland C, Pepke-Zaba J, Cremona G, Butt A Y, Large S R, Wells F C and Wallwork J. Impairment of endothelium dependent pulmonary artery relaxation in chronic obstructive lung disease. N. Engl. J. Med. 1991; 324:1539-1547.
8. Saetta M. Airway inflammation in chronic obstruction pulmonary disease. Am. J. Resp. Crit. Care Med. 1999; 160:517-S20.
9. Sanguinetti C M. Oxidant/antioxidant imbalance: role in the pathogenesis of COPD. Respiration 1992; 59(1):20-3.
10. Heidal J R, Fox R B, LeMarbe P A, Perri R and Repine J E. Altered oxidative metabolic responses in vitro of alveolar macrophages from asymptomatic cigarette smokers. Am. Rev. Respir. Dis. 1981; 123:85-89.
11. Mateos F, Brock J H and Perez-Arellano J L. Iron metabolism in the lower respiratory tract. Thorax 1998; 53:594-600.
12. Cross C E, O′Neill C A, Reznick A Z, Hu M L, Marcocci L, Packer L and Frei B. Cigarette smoker oxidation of human plasma constituents. Ann. N.Y. Acad. Sci. 1993; 686:72-90.
13. Cantin A and Crystal R G. Oxidants, antioxidants and the pathogenesis of emphysema. Eur. J. Respir. Dis. 1985; 66(139): 7-17.
14. Rahman I and MacNee W. Role of oxidants/antioxidants in smoking induced lung diseases. Free Radic. Biol. Med. 1996; 21:669-681.
15. MacNee W and Rahman I. Oxidants and antioxidants as therapeutic targets in COPD. Am. J. Respir. Crit. C are Med. 1999;160:S58-S65.
16. Rahman I, Morrison D, Donaldson K and MacNee W. Systemic oxidative stress in asthma, COPD and smokers. Am. J. Respir .Crit. Care Med. 1996; 154:1055-1060.
17. Turato G, Di Stefano A, Maestrelli P, Mapp C E, Ruggieri M P, Fabri L M and Saetta M. Effect of smoking cessation on airway inflammation in chronic bronchitis. Am. J. Resp. Crit. Care Med. 1995; 152:1262-1267.
18. Llewellyn Jones C G and Stockley R A. The effects of B₂ agonists and methyl xanthines on neutrophil function in vitro. Eur. Respir. J. 1994; 73:1460-1466.
19. Nielson C P, Crowley J J, Cusack B J and Vestal R E. Therapeutic concentrations of theophylline and enprofylline potentiate catecholamine effects and inhibit leukocyte activation. J. Allergy Clin. Immunol. 1986; 78:660-667.
20. Mofuard J, Manier G, Piller O and Castaign Y. Effect of inhaled nitric oxide on hemodyanamics and V_{A}/Q inequalities in patients with chronic obstructive pulmonary disease. Am. J. Resp. Crit. Care Med. 1994; 149:1482-87.
21. Cross C E, Halliwell B and Allen A. Antioxidant protection: a function of trcheobronchial and gastrointestinal mucus. Lancet 1984; 1:1328-1330.
22. Moldeus P, Cotgreave I A and Berggren M. Lung protection by a thiol containing antioxidant: N-acetyl cysteine. Respiration 1986; 50:31-42.
23. Renkema T E L Postma D S, Noordhoek J A, Sluster H J and Kauffman H F. Influence of in vivo prednisolone on increased in vitro O₂— generation by neutrophils in emphysema. Eur. Respir. J. 1993; 6:90-95.
24. Brantly M L, Paul L D, Miller B H et al. Clinical features and history of the destructive lung disease associated with α-1 antitrypsin deficiency of adults with pulmonary symptoms. Am. Rev. Respir. Dis. 1988; 138:327-336.
25. Ishii T, Matsuse T, Teramoto S, Matsui H, Miyao M, Hosoi T, Takahashi H, Matsui H and Ouchi Y. Glutathione-S-transferase P1 (GSTP1) polymorphism in patients with chronic obstructive pulmonary disease. Thorax 1999; 54:693-696.
26. Kaneko M, Suzuki K, Furui H, Takagi K and Satake T. Comparison of theophylline & enprofylline effects on human neutrophil superoxide production. Clin. Exp. Pharmacol. Physiol. 1990; 17:849-859.
27. Linden M, Wieslander E, Eklund A, Larson K and Brattsand R. Effects of oral N-acetyl cysteine on cell content and macrophage function in bronchoalveolar lavage from healthy smokers. Eur. Respir. J. 1988; 1:645-650.
28. Lomas D A, M. Ip, Chamba A and Stockley R A. The effect of in vitro and in vivo dexamethasone on human neutrophil function. Agents Actions 1991; 33:279-285.
29. DeBcuker W A, Decivera J, Van Overweld E J and Vermeire P A. The effects of treatment with inhaled corticosteroids on inflammation in stable chronic obstructive pulmonary disease (abstract). Am. J. Resp. Crit. Care Med 1996;153:A823.
30. Silverman E K, Pierce J A, Province M A et al. Variability of pulmonary function in alpha-1-antitrypsin deficiency:clinical correlates. Ann. Intern. Med. 1989; 111:982-991.

1-16. (canceled)

17. A method of determining a subject's predisposition to emphysema in chronic obstructive pulmonary disease (COPD), the method comprising:
detecting an allelic variant of an eNOS gene, the variant comprising allele -786 T, allele -786 C, allele 4B, or allele 4A.

18. The method of claim 17, wherein detecting allele -786 T, allele 4B, or both alleles indicates a decreased risk of emphysema.

19. The method of claim 17, wherein detecting allele -786 C, allele 4A, or both alleles indicates an increased risk of emphysema.

20. The method of claim 17, wherein detecting comprises:
amplifying a region of the subject's eNOS gene employing an oligonucleotide primer comprising a sequence of SEQ. ID NO. 3, SEQ. ID NO. 4, SEQ. ID NO. 5, or SEQ. ID NO. 6

21. The method of claim 20, comprising:
amplifying a region of the subject's eNOS gene employing oligonucleotide primers comprising the sequences of SEQ. ID NO. 3 and of SEQ. ID NO. 4.

22. The method of claim 20, comprising:
amplifying a region of the subject's eNOS gene employing oligonucleotide primers comprising the sequences of SEQ. ID NO. 5 and SEQ. ID NO. 6.

23. The method of claim 17, wherein detecting comprises:
amplifying DNA with a sequence from SEQ ID NO. 1 or SEQ ID NO. 2 and containing the polymorphic markers in the eNOS gene.

24. The method of claim 17, wherein detecting comprises employing a kit comprising:
a primer or probe comprising a sequence of SEQ ID No. 3, SEQ. ID NO. 4, SEQ. ID NO. 5, or SEQ. ID NO. 6;
buffer; and
instruction manual;
wherein the kit is suitable for identification of eNOS allelic variants to establish a subject's susceptibility to COPD.

25. The method of claim 24, wherein the kit further comprises:
restriction enzyme,
reverse transcriptase,
polymerase,
nucleoside triphosphate,
reagent effective to label the oligonucleotide,
buffer for reverse transcription, polymerase chain reaction, or hybridization reactions, or combination thereof.

26. The method of claim 25, wherein the label is biotin.

27. The method of claim 26, wherein the reagent effective to label the oligonucleotide comprises streptavidin enzyme conjugate, enzyme substrate, chromogen, or mixture thereof.

28. The method of claim 17, wherein the subject is a mammal.

29. The method of claim 17, wherein the subject is a human.
